(19) United States
(12) Patent Application Publication
(10) Pub. No.: US 2017/0340860 A1
(43) Pub. Date: Nov. 30, 2017

(54) WELDED ON CATHETER HUB

(71) Applicant: TERUMO MEDICAL CORPORATION, Somerset, NJ (US)

(72) Inventors: Mark Eberhardt, Elverson, PA (US); Eric Hearn, Middletown, DE (US)

(73) Assignee: TERUMO MEDICAL CORPORATION

(21) Appl. No.: 15/165,234

(22) Filed: May 26, 2016

(51) Int. Cl. A61M 25/00; B29C 65/14; A61M 25/06; B29C 65/68; B29L 31/00

(52) U.S. Cl. CPC A61M 25/0097; A61M 25/0662; B29C 65/68; B29C 65/1412; A61M 25/0014; A61M 25/0052; A61M 25/0023; B29L 2031/7542

(57) ABSTRACT

The disclosed technology includes a catheter or sheath assembly and a method for manufacturing same. The manufacturing process includes obtaining a catheter or sheath having a distal end and a proximal end, and obtaining a hub pre-formed separately from the catheter or sheath. The hub can include a pre-formed receptacle portion configured to receive a portion of the catheter or sheath. A portion of the catheter or sheath can be inserted into the pre-formed receptacle portion of the hub. The receptacle portion can be melted to a semi-fluid state and can be compressed against the portion of the catheter or sheath in the hub.

### FIELD OF THE TECHNOLOGY

The disclosed technology relates to the field of vascular access devices. In particular, the disclosed technology relates to sheath and catheter assemblies and systems and methods for manufacturing them.

### BACKGROUND OF THE TECHNOLOGY

In the medical field, many medical procedures require entry into a patient's blood or other vessel for purposes of accessing a desired treatment site to perform various procedures, e.g., surgical, treatment, or diagnostic procedures. To gain access to the desired site, a sheath or catheter is generally advanced through the vessel. Once in place within the patient's vessel, various types of medical instruments can be advanced through the sheath and positioned at the desired site so that the procedure may be performed.

To initially gain access to a particular site within a patient, a needle is used to puncture the patient's skin and gain entry to a desired vessel. A guide wire is then inserted into a lumen in the needle and is fed into the blood vessel. The needle is then removed, with the guide wire being left in place. A dilator/catheter assembly or dilator/sheath assembly is then placed over the guide wire and advanced to a position inside the blood vessel. Once the guidewire and the dilator/catheter assembly or dilator/sheath assembly is advanced within the blood vessel to the desired site, the dilator and guidewire are removed. The catheter or sheath can then be used to introduce and guide medical instrumentation to the desired site.

As the procedures are performed within a patient's body, instrument quality is of great concern. Part of this concern can be addressed by improvements in manufacturing. Accordingly, research and development for improving the quality of medical instruments and improving their manufacturing are areas of ongoing concern in the medical field.

### SUMMARY

Therefor there is a need for improved catheter and sheath design and manufacture that provides improved ease of manufacture and higher quality. As described herein various embodiments provide and improved catheter and sheath design.

In one embodiment, a catheter or sheath assembly is provided having a distal end and a proximal end wherein a catheter hub is pre-formed separately from the catheter. The catheter hub has a pre-formed receptacle portion for receiving a portion of the catheter being inserted into the pre-formed receptacle portion. The receptacle portion is re-formed into a molded portion by being melted to a semi-fluid state and mechanically compressed against the portion of the catheter inserted into the catheter hub. The outer surface of the portion of the catheter positioned in the catheter hub may also be melted and fused with the semi-fluid state of the molded portion catheter hub portion.

In further embodiments, the proximal portion of the catheter is wider than the remainder of the catheter (e.g., it is tapered or stepped). For example, the proximal portion may be widest at the proximal end and tapered from the proximal end to the remainder of the catheter. In this way, the proximal portion provides strain relief. The tapered or stepped catheter in such embodiments may having a constant inner diameter. In some embodiments, the proximal portion of the catheter may be partially inside the catheter hub and partially outside the catheter hub.

In yet further embodiments, the catheter may include a reinforcing coil along at least the proximal portion of the catheter. The reinforcing coil may be composed of a first winding pitched towards the proximal end and a second winding pitched away from the proximal end, the reinforcing coil being continuous between the first windings and the second windings and being turned between the first windings and the second windings.

In additional embodiments, the catheter may further include a band surrounding the reinforcing coil at approximately the proximal end of the catheter and holding the reinforcing coil from unwinding.

Various embodiments also include methods of manufacturing a catheter assembly described herein wherein the distal end of the catheter is inserted into the pre-formed receptacle portion of the catheter hub and the receptacle portion is melted to a semi-fluid state and the receptacle portion in the semi-fluid's state is compressed and deformed against the portion of the catheter in the catheter hub. In some embodiments, the outer surface of the catheter portion in the catheter hub is also melted and fused with the receptacle portion.

In various methods of manufacture, the receptacle portion is compressed by positioning a heat-shrinkable wrap around at least the pre-formed receptacle portion, and heated to a shrunken state to compresses the receptacle portion. In further embodiments, the catheter hub is then cooled to a solid state and the heat-shrinkable wrap in the shrunken state is removed from the catheter assembly.

While the above embodiments are discussed in the context of a catheter, it will be understood that the apparatus and method is equally extendable to sheath and sheath assemblies, and that wherever a catheter or sheath is referenced those terms can be used interchangeably in the context of this disclosure.

The benefits and advantages of the system and method disclosed herein have many uses. Overall these aspects and others described below provide a higher quality catheter and sheath assembly and method of manufacturing the same which is useful for a wide range of products.

### DESCRIPTION OF THE DRAWINGS

The foregoing and other aspects of the invention will be appreciated more fully from the following further description thereof and the accompanying drawings:

FIG. 1 is a diagram of an exemplary vascular access kit;

FIG. 2 is a diagram of an exemplary catheter or sheath assembly;

FIG. 3 is a diagram of a cross-sectional view of an exemplary catheter or sheath hub;

FIG. 4 is a diagram of a cross-sectional view of an exemplary coil-reinforced catheter or sheath;

FIG. 5 is a diagram of a cross-sectional view of the catheter or sheath assembly illustrated in FIG. 2;

FIG. 6 is a diagram of an exemplary system for assembling a sheath or catheter assembly;

FIG. 7 is a diagram of another exemplary system for assembling a sheath or catheter assembly;

FIG. 8 is a diagram of a cross-sectional view of the distal end of the catheter or sheath illustrated in FIG. 4;

FIG. 9 is a diagram of a cross-sectional view of the proximal end of the catheter or sheath illustrated in FIG. 4;

FIG. 10 is a diagram of exemplary windings at the proximal end of a coil-reinforced catheter or sheath;

FIG. 11 is a diagram of an exemplary proximal end of a coil-reinforced catheter or sheath with a band securing the proximal windings;

FIG. 12 is a flow diagram of an exemplary process of assembling a catheter or sheath assembly; and

FIG. 13 is a flow diagram of an exemplary process of assembling a catheter or sheath assembly using heat shrinkable tubing.

### DETAILED DESCRIPTION

The disclosed technology relates to sheath and catheter assemblies and systems and methods for manufacturing them.

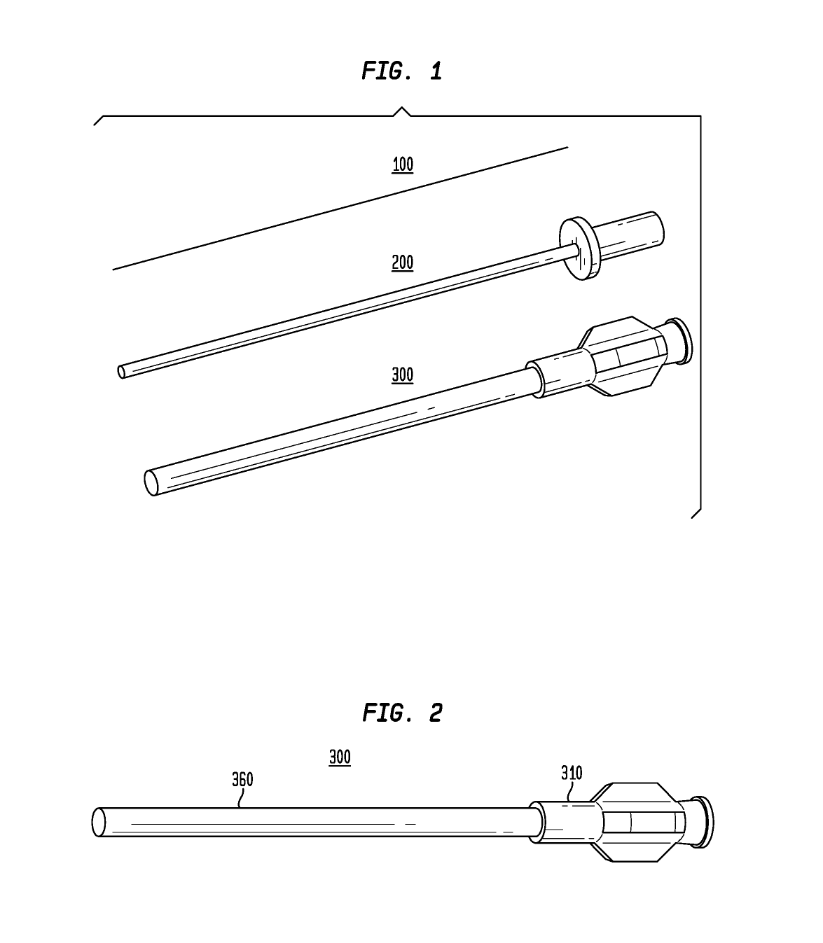

The process of accessing a patient's vasculature includes several steps. Referring to FIG. 1, there is shown an embodiment of a vascular access kit that includes a guidewire 100, a dilator assembly 200, and a sheath or catheter assembly 300. A needle (not shown) is used to puncture a patient's skin and is inserted into a vessel of the patient's vasculature. Then, a guidewire 100 is inserted into the lumen of the needle outside the patient and is advanced into the vessel of the patient through lumen, and the needle is withdrawn. In one procedure, the sheath/catheter assembly 300 is placed over the dilator assembly 200 outside the patient. The combined assembly is then introduced over the guidewire 100 outside the patient and is advanced along the guidewire 100 into the patient and the vessel. Then, the dilator 200 and guidewire 100 are removed from the patient, leaving the catheter or sheath 300 in place in the patient. Other kits and procedures for vascular access are known, and the disclosed technology applies to them as well. The access kit in FIG. 1 is merely exemplary and does not limit the scope of the disclosed technology.

Referring now to FIG. 2, there is shown an exemplary catheter assembly 300 or sheath assembly 300. For ease of reference, a catheter assembly 300 or sheath assembly will be referred to herein as a catheter assembly 300. It will be understood that although the disclosed technology may be described using the term “catheter”, the disclosed technology is applicable to sheath and sheath assemblies as well. The catheter assembly 300 illustrated in FIG. 2 includes a catheter hub 310 and a catheter 360. The illustrated shapes and dimensions are merely exemplary and do not limit the scope of the disclosed technology. In one embodiment, the catheter can be secured to the catheter hub and can be non-detachable from the sheath hub in the course of ordinary usage.

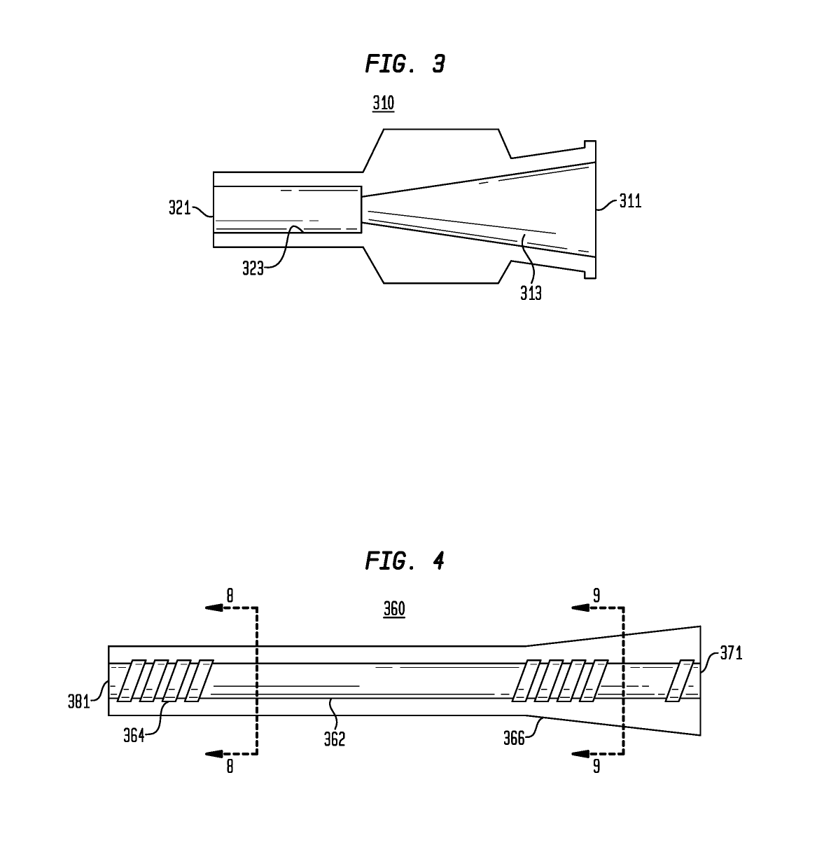

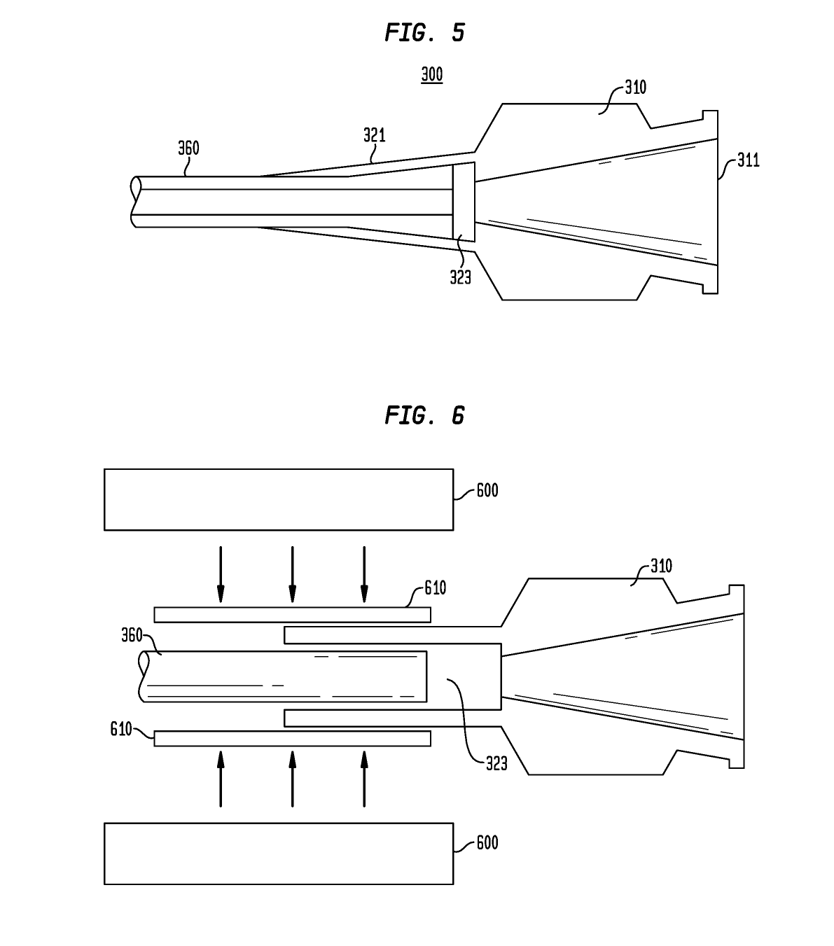

FIG. 3 is a diagram of a cross-sectional view of an exemplary catheter or sheath hub 310 in accordance with aspects of the disclosed technology. In accordance with one aspect of the disclosed technology, the illustrated hub 310 is pre-formed separately from a catheter or sheath and is intended to receive and secure a catheter or sheath 360. The hub 310 includes a proximal end 311 and a distal end 321. The proximal end 311 can receive a dilator or other medical instruments (not shown). The distal end 321 can receive a catheter or a sheath 360. The proximal portion 311 of the hub 340 can include a lumen 313 having a substantially frusto-conical shape that directs a dilator or other medical instrument towards the center of the hub and into a catheter or sheath. The distal portion 321 of the hub 310 can include a pre-formed receptacle 323 having a substantially cylindrical shape that can receive a sheath or a catheter 360. As described later herein in connection with FIGS. 5-7, the distal portion can be melted to a semi-fluid state and re-formed to secure the sheath or catheter therein. The illustrated shapes and dimensions are merely exemplary and do not limit the scope of the disclosed technology.

FIG. 4 is a diagram of a cross-sectional view of an exemplary coil-reinforced catheter or sheath 360. The catheter or sheath has a proximal end 371 and a distal end 381. In the illustrated embodiment, the catheter can include an inner tubing 362 that is surrounded by a coil 364, which in turn is surrounded by an outer tubing 366. The coil can traverse the entire length of the catheter, but for simplicity of illustration, not all of the coils are illustrated. In one aspect of the disclosed technology, the proximal portion 371 of the catheter 360 can have a diameter B that is greater than the diameter A of the distal portion 381 of the catheter 360. In one embodiment, the greater diameter B at the proximal portion 371 can be formed by thicker outer tubing. In one embodiment, the proximal portion 371 can include taper, as illustrated in FIG. 4. The greater diameter and the taper at the proximal portion 371 provides additional strength at the location where the catheter or sheath interacts with the hub 310. This location is where the catheter/sheath experiences strain, and the greater diameter and the taper at the proximal portion 371 can provide strain relief for the catheter/sheath.

FIG. 8 is a diagram of a cross-sectional view of the distal end 381 of the catheter or sheath illustrated in FIG. 4. The distal cross-section includes an inner tubing 362, a reinforcing coil 364, and an outer tubing 366. FIG. 9 is a diagram of a cross-sectional view of the proximal end 371 of the catheter or sheath illustrated in FIG. 4. The proximal cross-section includes the inner tubing 362 and the reinforcing coil 364, and the outer tubing 366 can be thicker at the proximal portion 371 to provide strain relief.

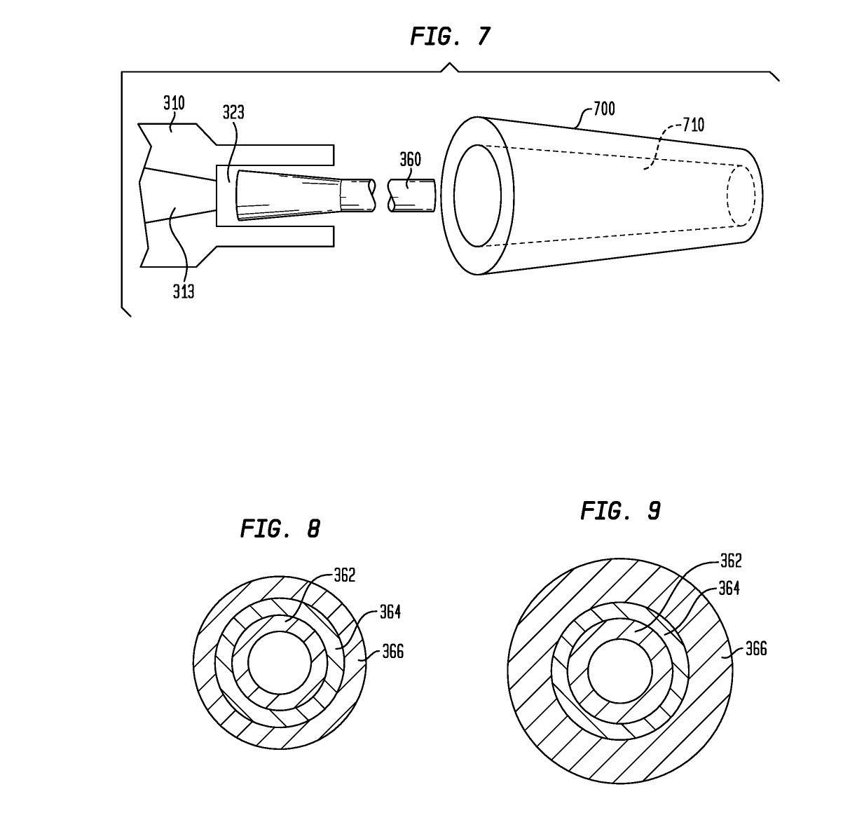

Referring now to FIG. 5, there is shown a diagram of a cross-sectional view of the catheter or sheath assembly 300. In accordance with one aspect of the disclosed technology, the pre-formed hub 310 has received a catheter or sheath 360, and the distal receptacle 323 of the hub 310 has been melted to a semi-fluid slate and re-formed to secure the sheath or catheter 360 therein. Exemplary systems for melting and re-forming the hub receptacle 323 of FIG. 5 are described in connection with FIG. 6 and FIG. 7.

Referring now to FIG. 6, there is shown a diagram of an exemplary system for assembling a sheath assembly or catheter assembly 300. The system can include a heat source 600 and a heat shrinkable tubing 610, such as a shrink-wrap. The heat source 600 can be any known source, such as, halogen or infrared heat sources. The heat shrinkable tubing 610 can be made from materials, such as, medical grade fluoropolymer, polyolefin or polyethylene terephthalate. In one aspect of the disclosed technology, the hub receptacle portion 323 can melt to a semi-fluid state when heated by the heat source 600, but the heat shrinkable tubing 610 does not melt when heated by the heat source 600. The heat shrinkable tubing 610 shrinks under the heat and compresses the semi-fluid hub receptacle portion 323 against the catheter/sheath 360. In one embodiment, a portion of the catheter or sheath 360 can also melt to a semi-fluid state from the heat of the heat source 600. In one embodiment, only the outer tubing 366 of the catheter or sheath 360 may melt. The semi-fluid hub receptacle portion 323 and the semi-fluid catheter/sheath 360 can be compressed together and their material can mix and bond. When the hub receptacle 323 and the catheter/sheath 360 are cooled, their mixed and bonded material can cause the catheter/sheath 360 to be secured within the hub 310. When the material have cooled, the shrink wrap 610 can be removed and discarded.

FIG. 7 is a diagram of another exemplary system for assembling a sheath or catheter assembly 300. As with FIG. 6, the hub receptacle portion 323 can melt to a semi-fluid state when heated by the heat source 600. In one embodiment, at least a portion of the catheter or sheath 360 can also melt to a semi-fluid state. Rather than using a shrink wrap 610 to compress the hub receptacle 323 against the catheter/sheath 360, as in FIG. 6, the system of FIG. 7 includes a structure 700 that includes a frusto-conical portion 710 that receives the hub and the catheter/sheath 360 and hub 310. As hub 310 is inserted into the frusto-conical portion 710, and the frusto-conical shape of the receptacle shapes the semi-fluid hub receptacle 323 and compresses it against the catheter or sheath 360. When the hub receptacle 323 and the catheter/sheath 360 are cooled, their mixed and bonded material can cause the catheter/sheath 360 to be secured within the hub 310.

What have been described above are sheath assemblies and catheter assemblies and systems and methods of manufacturing them. The illustrated and described embodiments, shapes, and dimensions are exemplary and do not limit the scope of the disclosed technology. The following paragraphs relate to and expand upon the coil-reinforced catheter or sheath illustrated in FIG. 4.

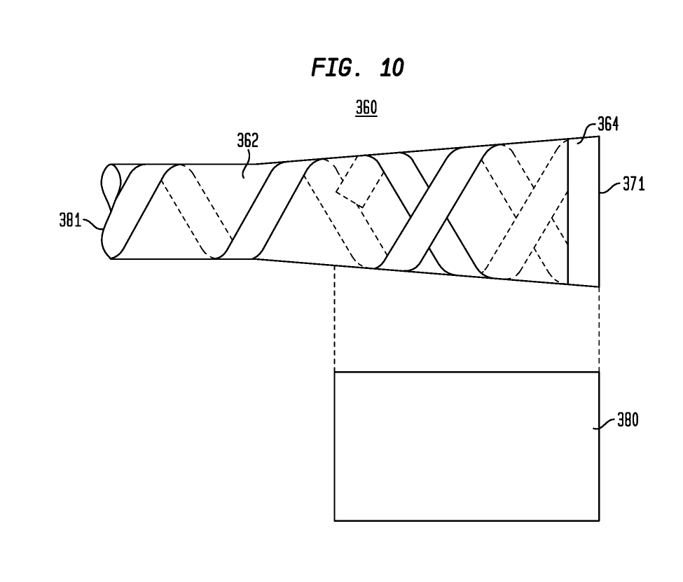

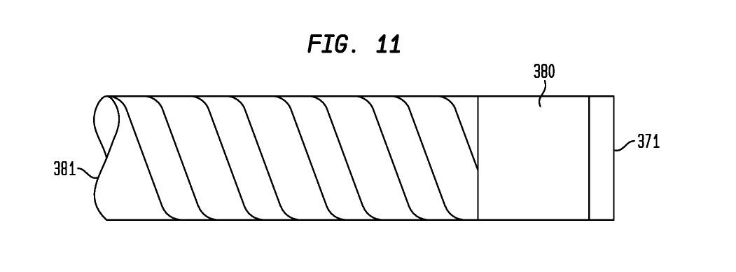

Referring now to FIG. 10, there is shown a diagram of exemplary windings at the proximal end of a coil-reinforced catheter or sheath. In one aspect of the disclosed technology, the reinforcing coil surrounding the inner tubing can include back-winding that secures the end of the coil 364 under itself. As shown in FIG. 10, the proximal end 371 of the catheter or sheath 360 is illustrated. The coil windings 364 can begin slightly inward from the proximal end 371 and can include a particular number of windings toward the proximal end 371 and then reverse direction and wind back over itself toward the distal end 381 of the catheter/sheath 360. The initial windings of the coil 364 are tucked underneath other windings and do not unravel. A band 380 can be placed over the proximal end 371 of the catheter/sheath 360 to ensure that the windings are secure and will not unravel. An exemplary illustration of the resulting proximal end 371 of the catheter or sheath 360 is shown in FIG. 11.

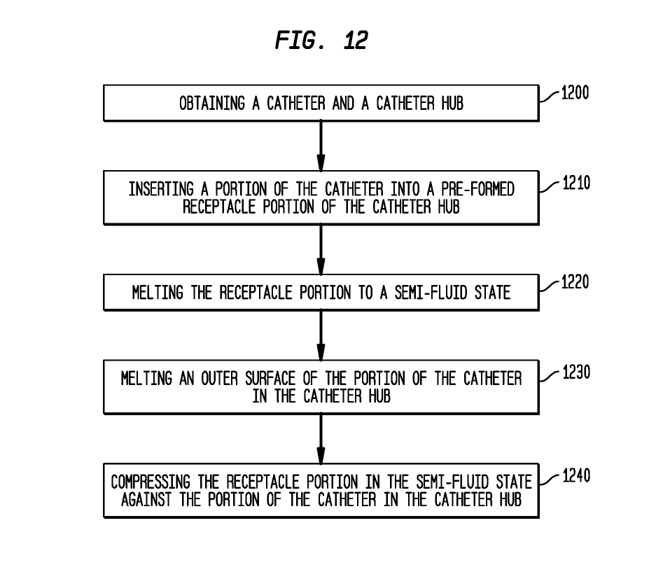

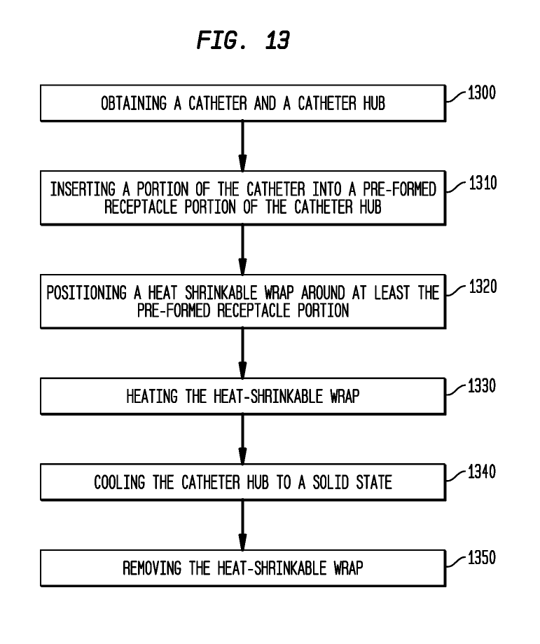

FIG. 12 and FIG. 13 show flow diagrams of exemplary methods of manufacturing a catheter assembly 300 or a sheath assembly, as described herein in accordance with the disclosed technology. Although the illustrations refer to catheter, the illustrated methods apply to sheaths as well.

Referring to FIG. 12, the disclosed technology including obtaining a catheter and a catheter hub pre-foil led separately from the catheter 1200, and inserting a portion of the catheter into a pre-formed receptacle portion of the catheter hub 1210. A heat source can melt the receptacle portion into a semi-fluid state 1220, and can melt an outer surface of the portion of the catheter that is inside the catheter hub 1230. The semi-fluid receptacle portion of the hub can be compressed against the portion of the catheter that is inside the hub, to secure the catheter within the hub 1240.

Referring to FIG. 13, the disclosed technology including obtaining a catheter and a catheter hub pre-formed separately from the catheter 1300, and inserting a portion of the catheter into a pre-formed receptacle portion of the catheter hub 1310. A heat shrinkable wrap can be positioned around at least the pre-formed receptacle portion 1320. A heat source can heat the heat-shrinkable wrap 1330, and can melt the receptacle portion into a semi-fluid state and melt an outer surface of the portion of the catheter that is inside the catheter hub. The shrinking of the heat shrinkable wrap compresses the semi-fluid receptacle portion of the hub against the portion of the catheter that is inside the hub, and secures the catheter within the hub. The catheter hub can be cooled to a solid state 1340, and the heat shinkable wrap can be removed 1350.

The embodiments described herein are merely illustrative and do not limit the scope of the disclosed technology. The disclosed technology may be embodied in other ways not explicitly described herein, without departing from the spirit thereof.

What is claimed is:

1. A catheter assembly, comprising;
a catheter having a distal end and a proximal end; and
a catheter hub pre-formed separately from the catheter, the catheter hub comprising a pre-formed receptacle portion, a portion of the catheter being inserted into the pre-formed receptacle portion, wherein the receptacle portion is re-formed into a molded portion by being melted to a semi-fluid state and mechanically compressed against the portion of the catheter in the catheter hub.

2. A catheter assembly as in claim 1, a proximal portion of the catheter being wider than a remainder of the catheter, the proximal portion being widest at the proximal end and tapering from the proximal end to the remainder of the catheter, the proximal portion providing strain relief, and the catheter having a constant inner diameter.

3. A catheter assembly as in claim 2, the proximal portion of the catheter being partially inside the catheter hub and partially outside the catheter hub.

4. A catheter assembly as in claim 1, wherein an outer surface of the portion of the catheter in the catheter hub is melted and fuses with the semi-fluid state of the molded portion.

5. A catheter assembly as in claim 1, the catheter comprising a reinforcing coil along at least a proximal portion of the catheter, the reinforcing coil comprising first windings pitched towards the proximal end and second winding pitched away from the proximal end, the reinforcing coil being continuous between the first windings and the second windings and being turned between the first windings and the second windings.

6. A catheter assembly as in claim 1, the catheter further comprising a band surrounding a reinforcing coil at approximately the proximal end of the catheter and holding the reinforcing coil from unwinding.

7. A method of manufacturing a catheter assembly, the method comprising:
obtaining a catheter having a distal end and a proximal end;
obtaining a catheter hub pre-formed separately from the catheter, the catheter hub comprising a pre-formed receptacle portion configured to receive a portion of the catheter;
inserting a portion of the catheter into the pre-formed receptacle portion of the catheter hub;
melting the receptacle portion to a semi-fluid state; and
compressing the receptacle portion in the semi-fluid state against the portion of the catheter in the catheter hub.

8. A method as in claim 7, wherein compressing the receptacle portion comprises:
positioning a heat-shrinkable wrap around at least the pre-formed receptacle portion, and heating the heat-shrinkable wrap to a shrunken state that compresses the receptacle portion.

9. A method as in claim 8, further comprising:
cooling the catheter hub to a solid state; and
removing the heat-shrinkable wrap in the shrunken state from the catheter assembly.

10. A method as in claim 7, further comprising melting an outer surface of the portion of the catheter in the catheter hub, the outer surface fusing with the receptacle portion in the semi-fluid state.

11. A sheath assembly, comprising;
a sheath having a distal end and a proximal end; and
a sheath hub pre-formed separately from the sheath, the sheath hub comprising a pre-formed receptacle portion, a portion of the sheath being inserted into the pre-formed receptacle portion, wherein the receptacle portion is re-formed into a molded portion by being melted to a semi-fluid state and mechanically compressed against the portion of the sheath in the sheath hub.

12. A sheath assembly as in claim 11, a proximal portion of the sheath being wider than a remainder of the sheath, the proximal portion being widest at the proximal end and tapering from the proximal end to the remainder of the sheath, the proximal portion providing strain relief, and the sheath having a constant inner diameter.

13. A sheath assembly as in claim 12, the proximal portion of the sheath being partially inside the sheath hub and partially outside the sheath hub.

14. A sheath assembly as in claim 11, wherein an outer surface of the portion of the sheath in the sheath hub is melted and fuses with the semi-fluid state of the molded portion.

15. A sheath assembly as in claim 11, the sheath comprising a reinforcing coil along at least a proximal portion of the sheath, the reinforcing coil comprising first windings pitched towards the proximal end and second winding pitched away from the proximal end, the reinforcing coil being continuous between the first windings and the second windings and being turned between the first windings and the second windings.

16. A sheath assembly as in claim 11, the sheath further comprising a band surrounding a reinforcing coil at approximately the proximal end of the sheath and holding the reinforcing coil from unwinding.

17. A method of manufacturing a sheath assembly, the method comprising:
obtaining a sheath having a distal end and a proximal end;
obtaining a sheath hub pre-formed separately from the sheath, the sheath hub comprising a pre-formed receptacle portion configured to receive a portion of the sheath;
inserting a portion of the sheath into the pre-formed receptacle portion of the sheath hub;
melting the receptacle portion to a semi-fluid state; and
compressing the receptacle portion in the semi-fluid state against the portion of the sheath in the sheath hub.

18. A method as in claim 17, wherein compressing the receptacle portion comprises:
positioning a heat-shrinkable wrap around at least the pre-formed receptacle portion, and
heating the heat-shrinkable wrap to a shrunken state that compresses the receptacle portion.

19. A method as in claim 18, further comprising:
cooling the sheath hub to a solid state; and
removing the heat-shrinkable wrap in the shrunken state from the sheath assembly.

20. A method as in claim 17, further comprising melting an outer surface of the portion of the sheath in the sheath hub, the outer surface fusing with the receptacle portion in the semi-fluid state.
